(19) United States
(12) Patent Application Publication
(10) Pub. No.: US 2002/0045845 A1
(43) Pub. Date: Apr. 18, 2002

(54) Bseat-support device

(76) Inventor: Jobst Kruttke, Hamburg (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 09/942,067

(22) Filed: Aug. 29, 2001

(30) Foreign Application Priority Data
Aug. 31, 2000 (DE) 20015071.5

(51) Int. Cl. A61F 5/00

(52) U.S. Cl. 602/32

(57) ABSTRACT

A support device for back and knees comprising a belt or loop that can be fitted around one's body while in a sitting position.

This invention relates to a support for back and knees. After standing up for a long time or walking for long distances, we often look for a bench or something equivalent to sit on for rest. Often, portable chairs are brought along whenever we enjoy the outdoors at a park, a concert, a parade, or any other air show. However, it is often problematic to transport or carry portable chairs due to their considerable weight and big size. Further, portable chairs take up significant space when unfolded, which may make it difficult in crowded areas to find an adequate space to place the chair.

In such situations, a visitor has no alternative, but either to stand or to sit down on the ground or grass. Sitting down directly on a flat surface for long periods of time without a back rest may however cause back pains and may be straining for one's legs and knees.

When sitting on the ground without a backrest, sitting position is often used, where one's feet are rested on the ground, while the knees are positioned upwards and supported by one's arms. However, this position may also cause arms and back pains.

An object of the present invention is to provide a support for one's back that may be used in a specific sitting position. This support has a minimal storage space and may be carried by anyone, including children and elderly. Further, this support is easy to use and inexpensive.

Further features and advantages of the invention will be apparent from a preferred embodiment, by way of example only, which is shown in the drawings and described in detail in the description below.

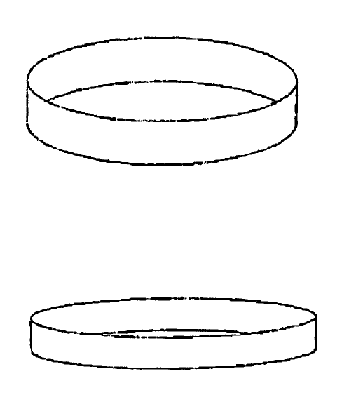

FIG. 1 illustrates the “bseat” support according to the present invention; and

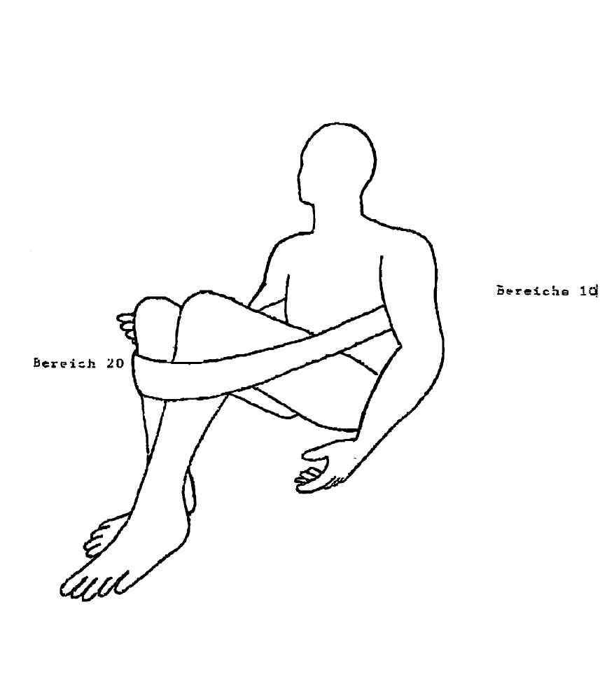

FIG. 2 illustrates one manner of using the “bseat” support of FIG. 1.

According to the present invention the “bseat” support is a belt or loop that can be fitted around one's body while in a sitting position.

As illustrated in FIG. 1, the belt support is a loop of approximately one centimeter to fifteen centimeters in width and of length proportional to the size of an individual using the loop. The length of the loop may be either fixed such as “one size only” so only one size may be provided or the length of the loop may be adjustable.

It will be appreciated that there are multiple ways of making the length of the loop or belt adjustable. For example, the adjustment of the loop length may be achieved by providing a belt buckle and corresponding holes in the loop. Further, adjustment of length may be accomplished by means of an adhesive or velcro system.

According to one embodiment of the present invention, the “bseat” support device may be fitted around one's back and knees. The optimal sitting position is illustrated in FIG. 2. In this position the knees of the user point upwards. The “bseat” support device is placed over the head, such that a region 10 of the “bseat” support device may be arranged at a desired location around the back or lumbar region. A second portion 20 of the “bseat” support device may be then positioned over one's knees. The “bseat” support device may then be adjusted in length in such a way that the “bseat” fits rigidly over the knees and back. The back region is thus supported by the pulling force exerted by the knees. In this way, the lumbar region is relaxed and back aches are relieved. It should be noted that in order to properly support one's lower back, the “bseat” support device must be elongated to its maximum length such that the loop does not slip out of the desired position. Further, one may choose any comfortable knee position, such as having the knees slightly pulled apart, as illustrated in FIG. 2. One may also have the knees crossed.

The “bseat” support device may be manufactured from any textile material or similar material that may be easily handled and folded. According to a preferred embodiment of the present invention, the support material is inelastic, such that the “bseat” support device actually fits rigidly around one's back and knees.

1. Support device for back and knees comprising a belt or loop that can be fitted around one's body while in a sitting position.

2. Support device for back and knees according to claim 1, characterized in that the belt or loop comprises two portions (10, 20), wherein the first region (10) is arranged in one's back and the second region is arranged around one's knees.

3. Support device for back and knees according to claim 1 or 2, further comprising adjustment means for adjusting the belt or loop length.

4. Support device for back and knees according to claim 3, wherein the adjustment means for adjusting the belt or loop length is a buckle and corresponding holes in the belt or loop.

5. Support device for back and knees according to claim 3, wherein the adjustment means for adjusting the belt or loop length is by means of an adhesive or velcro system.

6. Support device for back and knees according to one of the preceding claims, wherein the belt or loop is manufactured from any textile material or similar material.

7. Support device for back and knees according to one of the preceding claims, wherein the belt or loop is manufactured from an inelastic material.
